(19) United States
(12) Patent Application Publication
(10) Pub. No.: US 2018/0207414 A1
(43) Pub. Date: Jul. 26, 2018

(54) NAIL PENETRATION DEVICE FOR DELIVERY OF ANTI-FUNGAL TREATMENTS

(71) Applicant: Adolfo Napolez, Flossmoor, IL (US)

(72) Inventor: Adolfo Napolez, Flossmoor, IL (US)

(21) Appl. No.: 15/877,759

(22) Filed: Jan. 23, 2018

Related U.S. Application Data
(60) Provisional application No. 62/449,245, filed on Jan. 23, 2017.

(51) Int. Cl. A61M 35/00; A61B 17/3205

(52) U.S. Cl. CPC A61M 35/003; A61B 2017/00747; A61B 17/32053

(57) ABSTRACT

The present invention provides a nail penetration device for delivery of an antifungal treatment. Also provided are methods for introducing an antifungal treatment to an infected tissue, along with nail fungus treatment kits. Such a device is useful for treatment of fungal infection.

### CROSS-REFERENCE TO RELATED APPLICATION

This application claims the benefit of U.S. Provisional Application No. 62/449,245, filed on Jan. 23, 2017, the disclosure of which is incorporated by reference in its entirety.

### FIELD OF THE INVENTION

The subject matter disclosed herein is generally directed to treatment of fungal infection. In particular, the invention relates to a device for delivery of anti-fungal treatments.

### BACKGROUND

Toenail fungus is a ubiquitous condition both in the U.S. and worldwide. Approximately 35-40 million people in the U.S. suffer with toenail fungus of either one or multiple nails. Presently, 2-18% of people worldwide suffer from toenail fungus infections, representing approximately 800 million people. Toenail fungus is a disease of predominantly the adult population. While only 1 out of 200 people under the age of 18 are infected, in the adult population, approximately 48% of people have at least one toe affected by fungus by the age of 70 years.

Current treatments for toenail fungus include nail trimming, oral prescription medication, topical medication, nail removal, and laser therapy. While these methods can help with infection, there are drawbacks to each option. Nail trimming done by a podiatrist can be costly and inefficient. Oral medication requires a prescription, and an infection will likely require multiple prescriptions. Topical therapy often does not reach the fungal site and is usually ineffective. Complete nail removal is painful and expensive. Laser therapy is also expensive, ranging from $750 to $1500 per person, depending on the number of nails being treated and typically takes anywhere from 3-10 treatments to cure infection. Insurance is often also a consideration, as treatments for toenail fungus is not covered by most insurance policies. Presently, the typical cure rate for nail fungus eradication is less than 10%, so obviously a new method of treatment is required.

### SUMMARY OF THE INVENTION

In one aspect, the invention provides a nail penetration device for delivery of an antifungal treatment to a subject in need thereof, comprising: a cap having a circular blade at the distal end, and threading at the opposite end as a means for attachment to a container; and a container having threading at the open end for attachment to the cap. In one embodiment, the circular blade is 3 mm in diameter. In another embodiment, the circular blade is hollow and has a thickness of 1 mm. In another embodiment, the circular blade is hollow and has an outer diameter of 3 mm and an inner diameter of 1 mm. In still another embodiment, the circular blade has a height of 2 mm. Further embodiments provide a circular blade comprising a sharp cutting surface on the outer diameter of the blade.

In another embodiment, the cap has a length of 2 cm. In further embodiments, the cap is made of plastic, or the plastic is non-compressible. In another embodiment, the cap has a distal end smaller in diameter than the opposite end. In another embodiment, the circular blade is comprised at the distal end. In further embodiments, the cap is made of plastic. In another embodiment, the cap and the container comprise threading as a means for attaching. In further embodiments, the container is a bottle, or the bottle is compressible, or the bottle is made of plastic.

In another aspect, the invention provides a method for providing an antifungal treatment to an infected tissue comprising the steps of: attaching a cap to a container by use of threading to screw the cap onto the distal end of the container, wherein the container comprises in its interior an antifungal treatment; inverting the assembled device and contacting the circular blade at the distal end of the device with the surface of a nail infected with a fungus; rotating the device to introduce a hole in the infected nail with the circular blade; and compressing the container to dispense the antifungal treatment into the hole in the nail. In one embodiment, multiple holes are introduced to the nail infected with a fungus. In another embodiment, the white part of the nail is avoided.

In another aspect, the invention provides a nail fungus treatment kit comprising: a cap having a circular blade at the distal end, and threading at the opposite end as a means for attachment to a container; and a container having threading at the open end for attachment to the cap. In one embodiment, such a kit further comprises an antifungal treatment or medication. In another embodiment, such a kit further comprises a package, said cap and said container being removably enclosed within said package.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

FIG. 1—Shows a side view of a nail penetration device for delivery of antifungal treatments. Depicted are a circular blade in a cap, which is screwed on to a compressible plastic bottle containing a fungal solution.

FIG. 2—Shows a top view of a circular blade.

FIG. 3—Shows a side view of a plastic bottle cap containing a circular blade.

FIG. 4—Shows a schematic illustration of a nail indicating holes made using a nail penetration device for delivery of antifungal treatments.

### DRAWING ELEMENTS

10 3 mm diameter circulator blade
11 threading of screw-on cap for attaching to compressible plastic container
12 compressible plastic container
13 2 cm cap containing the 3 mm diameter circular blade
14 threading on outer surface of container
15 antifungal solution
20 sharp cutting surface on outer rim of 3 mm circular blade
21 1 mm thickness of wall of circular blade
22 3 mm outer diameter of circular blade
23 1 mm inner diameter of circular blade
30 plastic cap made of hard, non-compressible plastic
31 2 mm height of circular blade in tip of cap
32 3 mm diameter of circular blade
33 2 mm height of circular blade
40 white part of nail from which healthy nail grows
41 holes in pink part of infected nail showing distribution of antifungal medicine
42 pink part of nail in which holes are made with circular blade

### DETAILED DESCRIPTION OF THE INVENTION

The device of the present invention delivers antifungal medication directly to the fungus at the site of infection by creating small, 3-mm holes in the infected nail, which allows for the application of daily treatment for a necessary duration. Treatment may be needed for 2-3 months, or any length of time necessary for elimination of the fungus and for regrowth of the nail to eliminate the access areas or holes created in the nail. Treatment as described herein may increase the eradication rate of nail fungus to nearly 75% per nail penetrated.

The present invention can be understood more readily by reference to the following detailed description, examples, drawings, and claims, and their previous and following description. However, before the present devices, methods, and/or kits are disclosed and described, it is to be understood that this invention is not limited to the specific devices, methods and/or kits disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an,” and “the” comprise plural referents unless the context clearly dictates otherwise. Ranges can be expressed herein as from “about” one particular value, and/or to “about” another particular value. When such a range is expressed, another aspect comprises from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

In simplest terms, disclosed herein are nail penetration devices for delivery of an antifungal treatment to an infected nail. In one aspect, the nail penetration devices disclosed herein comprise a circular blade comprised within a plastic cap that is screwed on to a bottle containing an antifungal treatment. Such a device is useful for producing small holes in a nail infected with a fungus such that an antifungal treatment may be placed in direct contact with the infected tissue and thus with the fungus underlying the nail. Although the device may be used for other purposes, it is discussed in the context of treatment of a nail infected with a fungus.

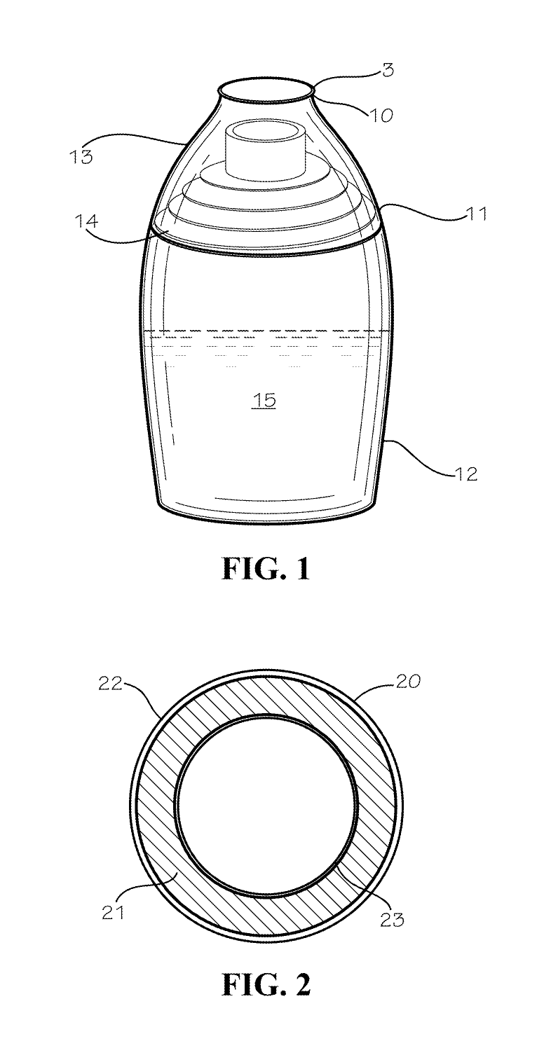

A view of one embodiment of the nail penetration device is illustrated. Referring to FIG. 1, the invention provides a nail penetration device for delivery of an antifungal treatment to a subject in need thereof, comprising: a cap 13 having a circular blade 10 at the distal end, and threading 11 at the opposite end as a means for attachment to a container 12; and a container 12 having threading 14 at the open end for attachment to the cap 13. In some embodiments, the circular blade 10 may be embedded within the plastic cap 13, and the plastic cap 13 is screwed on to a compressible plastic bottle 12 by threading 11 in the plastic cap 13. The circular blade 10 should be sharp enough to create 3-5 nail penetrations per container use. The holes or penetrations may be introduced only in the pink segment of the nail, thereby not damaging the white area of the nail that is responsible for continued nail growth.

In some embodiments, the circular blade 10 has a diameter of 3 mm, and the cap 13 containing the circular blade 10 has a height of 2 cm. In another embodiment, the circular blade 10 is hollow and has a thickness of 1 mm. In some embodiments the hollow circular blade 10 has an outer diameter of 3 mm and an inner diameter of 1 mm. In accordance with the invention, the circular blade 10 may have a height of 2 mm. In other embodiments, the circular blade 10 may have a sharp cutting surface on the outer diameter of the blade.

The cap 13 containing the circular blade 10 may have a length of 2 cm. In further embodiments, the cap 13 is made of plastic, such as a non-compressible plastic. One of skill in the art will recognize that other materials may also be used in accordance with the invention. In another embodiment, the cap 13 has a distal end smaller in diameter than the opposite end. In another embodiment, the circular blade 10 is comprised at the distal end. In another embodiment, the cap 13 and the container 12 comprise threading as a means for attaching. Other means of attaching the cap 13 to the container 12 may be used as appropriate with no alternation in function. In further embodiments, the container 12 is a bottle, and may be compressible to enable dispensing of the antifungal solution or medication 15. The container or bottle 12 may be made of plastic or may be made of another suitable material as appropriate in accordance with the invention. As one skilled in the art will appreciate, the components as described herein may be of any size as long as adequate for the assigned task, that being the production of small holes in a nail infected with a fungus.

The process of assembling the nail penetration device of the present invention is as follows. An antifungal solution 15 is placed within the compressible plastic container 12 and the plastic cap 13 is screwed on to the compressible plastic container 12 with the threading 11 on the plastic cap 13. The threading 11 on the plastic cap 13 is combined with the threading 14 on the outer surface of the plastic container 12 to attach the cap 13 to the container 12. The device is used with the cap 13 screwed onto the antifungal solution container or bottle 12 for better control. The assembled bottle and cap is then inverted to place the circular blade 10 into contact with an infected nail. The assembled bottle and cap are then used to introduce small holes in the nail. After the holes are created, the cap may then be removed, and the antifungal solution or treatment may then be dispersed into the sites created in the nail. In other embodiments, the compressible plastic bottle 12 may be squeezed or compressed to dispense the antifungal solution 15 or medication into the hole made with the circular blade 10 without removing the cap. In this way, the antifungal solution 15 is provided to the infected tissue or region. The cap and blade may be rinsed clean after each use with tap water.

Some embodiments provide for disposable use of the device as described herein. Other embodiments may provide a sterilizable cap and container such that the components may be sterilized with, for example, an autoclave between uses.

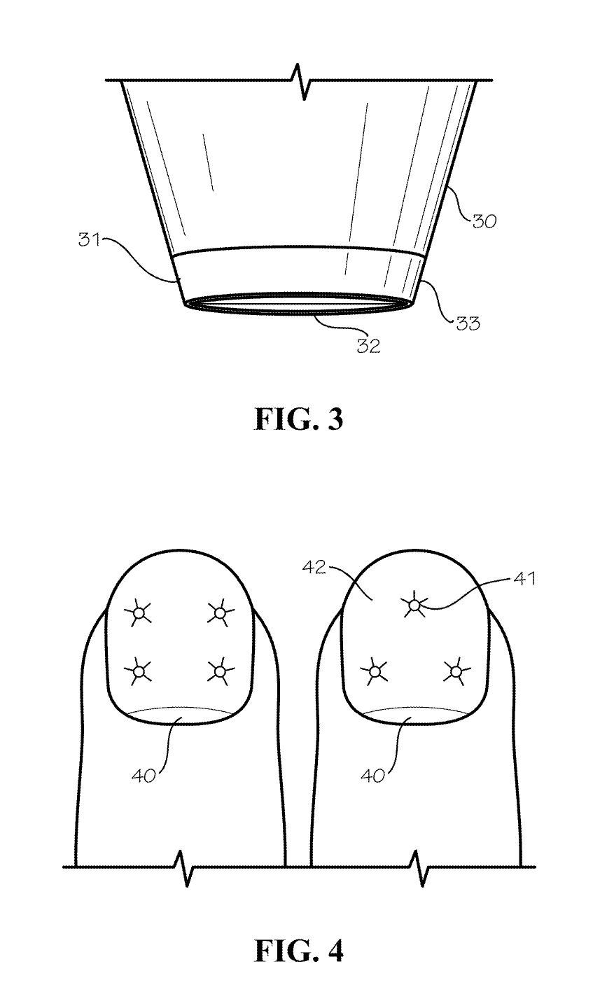

Referring to FIG. 2, the circular blade of the present device is hollow with an outer sharp cutting surface 20 having a diameter 22 of 3 mm. The circular blade has a thickness 21 of 1 mm. In another embodiment, the circular blade is hollow and has a thickness 21 of 1 mm. In some embodiments the hollow circular blade has an outer diameter 22 of 3 mm and an inner diameter 23 of 1 mm. In other embodiments, the circular blade may have a sharp cutting surface 20 on the outer diameter of the blade. Referring to FIG. 3, the circular blade 31 has a height 33 of 2 mm and is embedded in a bottle cap 30 made of hard, non-compressible plastic, and a diameter 32 of 3 mm. In accordance with the invention, the circular blade 10 may have a height of 2 mm.

Referring to FIG. 4, a nail penetration device as described herein may be used to produce 3 mm holes 41 in the pink part 42 of an infected nail. The nail penetration device enables direct treatment of infected tissue under the nail without the need for removal of the nail, and without damaging the ability of the nail to regrow by avoiding the white part of the nail 40.

In another aspect, the invention provides a method for providing an antifungal treatment to an infected tissue comprising the steps of: attaching a cap to a container by use of threading to screw the cap onto the distal end of the container, wherein the container comprises in its interior an antifungal treatment; inverting the assembled device and contacting the circular blade at the distal end of the device with the surface of a nail infected with a fungus; rotating the device to introduce a hole in the infected nail with the circular blade; and compressing the container to dispense the antifungal treatment into the hole in the nail. To create the holes in the nail, a clockwise and counterclockwise repetitive motion may be used, making typically 2-4 holes in an infected nail, directly overlying the fungus. The described method of introducing temporary holes in the affected nail, a fungal infection may be treated daily for approximately 2 months, the average time for a nail to grow out. In this way, the number of days the antifungal solution is directly in contact with the fungus is increased, thereby increasing the rate of cure. The nail penetrator described herein provides no more risk to a subject or patient, nor is it any more likely to cause injury, than other items available in the art for daily use, such as knives, scissors, tweezers, toenail clippers, and the like.

In some embodiments, multiple holes may be introduced to a nail infected with a fungus in order to maximize the contact of the antifungal solution or medication to the infected tissue or area. In another embodiment, the white part of the nail is avoided in order to prevent damage to the nail.

The method described for producing one or more holes in a nail infected with a fungus may be performed by any medical personnel, including moderately-skilled medical personnel, such as by a podiatrist, a medical assistant, an emergency room physician, or the like, without the need for surgery or a hospital stay, which will reduce the costs to the patient and/or the patient's insurer. In some embodiments, the present nail penetrator device is designed for personal use, for example for use at home without the need for medical personnel.

In another aspect, the invention provides a nail fungus treatment kit comprising: a cap having a circular blade at the distal end, and threading at the opposite end as a means for attachment to a container; and a container having threading at the open end for attachment to the cap. In one embodiment, such a kit may further comprise an antifungal treatment or medication, or the antifungal medication or solution may be provided separately. In some embodiments, such a kit may further comprise a package, wherein the cap and the container may be removably enclosed within said package. This may enable sterile transport and handling to eliminate or minimize contamination.

The nail penetration device disclosed herein may have many uses known to those of skill in the art. For example, the disclosed nail penetration device may be used to treat a fungal infection in any nail. Other embodiments may provide the use of alternate medications or compositions to treat other conditions, microbes, bacterial or fungal infections as appropriate for use in accordance with the invention.

As can be easily understood from the foregoing, the basic concepts of the present invention may be embodied in a variety of ways. The invention involves numerous and varied embodiments of a nail penetrator device and methods of using same. While the present invention has been described with respect to the preferred embodiments thereof, it will be recognized by those of ordinary skill in the art that many changes and modifications may be made without departing from the spirit and scope of the invention.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

As such, the particular embodiments or elements of the invention disclosed by the description or shown in the figures accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

Thus, the Applicant should be understood to claim at least: (i) the nail penetrator device herein disclosed and described, (ii) the related methods disclosed and described, (iii) similar, equivalent, and even implicit variations of each of these devices and methods, (iv) those alternative embodiments which accomplish each of the functions shown, disclosed, or described, (v) those alternative designs and methods which accomplish each of the functions shown as are implicit to accomplish that which is disclosed and described, (vi) each feature, component, and step shown as separate and independent inventions, (vii) the applications enhanced by the various systems or components disclosed, (viii) methods and apparatuses substantially as described herein and with reference to any of the accompanying examples, and (ix) the various combinations and permutations of each of the previous elements disclosed.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent non-provisional, continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent non-provisional continuation, division, or continuation-in-part application thereof or any reissue or extension thereon.

The claims set forth below are intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any non-provisional, continuation, division, or continuation-in-part, or similar application.

What is claimed is:

1. A nail penetration device for delivery of an antifungal treatment to a subject in need thereof, comprising:
a cap having a circular blade at the distal end, and threading at the opposite end as a means for attachment to a container; and
a container having threading at the open end for attachment to the cap.

2. The nail penetration device of claim 1, wherein the circular blade is 3 mm in diameter.

3. The nail penetration device of claim 1, wherein the circular blade is hollow and has a thickness of 1 mm.

4. The nail penetration device of claim 1, wherein the circular blade is hollow and has an outer diameter of 3 mm and an inner diameter of 1 mm.

5. The nail penetration device of claim 1, wherein the circular blade has a height of 2 mm.

6. The nail penetration device of claim 1, wherein the circular blade comprises a sharp cutting surface on the outer diameter of the blade.

7. The nail penetration device of claim 1, wherein the cap has a length of 2 cm.

8. The nail penetration device of claim 1, wherein the cap is made of plastic.

9. The nail penetration device of claim 8, wherein the plastic is non-compressible.

10. The nail penetration device of claim 1, wherein the cap has a distal end smaller in diameter than the opposite end.

11. The nail penetration device of claim 1, wherein the circular blade is comprised at the distal end.

12. The nail penetration device of claim 1, wherein the cap is made of plastic.

13. The nail penetration device of claim 1, wherein the cap and the container comprise threading as a means for attaching.

14. The nail penetration device of claim 1, wherein the container is a bottle.

15. The nail penetration device of claim 14, wherein the bottle is compressible.

16. The nail penetration device of claim 1, wherein the bottle is made of plastic.

17. A method for providing an antifungal treatment to an infected tissue comprising the steps of:
attaching a cap to a container by use of threading to screw the cap onto the distal end of the container, wherein the container comprises in its interior an antifungal treatment;
inverting the assembled device and contacting the circular blade at the distal end of the device with the surface of a nail infected with a fungus;
rotating the device to introduce a hole in the infected nail with the circular blade; and
compressing the container to dispense the antifungal treatment into the hole in the nail.

18. The method of claim 17, wherein multiple holes are introduced to a nail infected with a fungus.

19. The method of claim 18, wherein the white part of the nail is avoided.

20. A nail fungus treatment kit comprising:
a cap having a circular blade at the distal end, and threading at the opposite end as a means for attachment to a container; and
a container having threading at the open end for attachment to the cap.

21. The nail fungus treatment kit of claim 20, further comprising an antifungal treatment or medication.

22. The nail fungus treatment kit of claim 20, further comprising a package, said cap and said container being removably enclosed within said package.
